(19) United States
(12) Patent Application Publication
(10) Pub. No.: US 2009/0104291 A1
(43) Pub. Date: Apr. 23, 2009

(54) WATER-DISPERSIBLE NANOPARTICLE WHICH CONTAINS BLOOD CIRCULATION PROMOTER

(75) Inventor: Katsuhiko KANAZAWA, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/252,589

(22) Filed: Oct. 16, 2008

(30) Foreign Application Priority Data
Oct. 19, 2007 (JP) 2007-272386

(51) Int. Cl. A61K 47/42; A61K 31/506; A61K 31/44; A61K 36/51

(52) U.S. Cl. 424/725; 514/275; 514/773; 514/774; 514/776; 514/775; 514/356; 977/773

(57) ABSTRACT

It is an object of the present invention to provide a nanoparticle which comprises a blood circulation promoter and a biodegradable polymer, which is safe and excellent in terms of dispersion stability and has high transparency and good absorbability due to its small particle size. The present invention provides a water-dispersible nanoparticle which comprises a blood circulation promoter and a biodegradable polymer.

### FIELD OF THE INVENTION

The present invention relates to water-dispersible nanoparticles. More specifically, the present invention relates to water-dispersible nanoparticles which are excellent in dispersion stability and contain a blood circulation promoter.

### BACKGROUND ART

Extensive applications of fine particle materials have been expected for biotechnology. In particular, the application of nanoparticle materials generated based on the advancement of nanotechnology to food, cosmetics, pharmaceutical products and the like has been actively discussed. In this regard, the results of many studies have been reported.

For instance, regarding cosmetics, more obvious skin-improving effects have been required in recent years. Manufactures have been attempting to improve the functionality and usability of their own products and to differentiate their own products from competitive products by applying a variety of new technologies such as nanotechnology. In general, the stratum corneum serves as a barrier for the skin. Thus, medicines are unlikely to permeate therethrough into the skin. In order to obtain sufficient skin-improving effects, it is essential to improve the skin permeability of active ingredients. In addition, it is difficult to formulate many active ingredients due to poor preservation stability or tendency to result in skin irritancy, although they are highly effective to the skin. In order to solve the above problems, a variety of fine particle materials have been under development for the improvement of transdermal absorption and preservation stability, reduction of skin irritancy, and the like. Recently, a variety of fine particle materials such as ultrafine emulsions and liposomes have been studied (e.g., Mitsuhiro Nishida, Fragrance Journal, Nov. 17, (2005)).

Hitherto, it has been usual to add oil-based components to water-based cosmetics. However, since oil-based components are water-insoluble or weakly water-soluble, it has been common to mix an oil-based component, which is a so-called emulsified product, into an aqueous medium with the use of a certain emulsifying means. Light scattering of emulsified products depends on particle size. Thus, in some cases, emulsified products and foods or cosmetics containing emulsified products have cloudy appearances, which is not preferable. Therefore, it has been desired to miniaturize the particle size of an emulsified product to such an extent that the light scattering intensity becomes very low. In addition, emulsified products are generally in a metastable state. In such state, the particle size increases during storage and long-term storage results in separation, which are seriously problematic. In the cases of beverages, adherence of an aggregate of oil droplets to container walls and neck ring formation with such an aggregate are examples of oil droplet separation phenomenon observed in emulsified products.

As described above, many fine particle materials used for foods or cosmetics are related to emulsified products. Meanwhile, in recent years, polymer micelles have been gaining attention in the fields of pharmaceutical products and cosmetics (e.g., JP Patent Publication (Kokai) No. 2002-308728 A). Polymer micelles are characterized by large drug contents, high water solubility, high structural stability, non-accumulative properties, functional separation properties, and the like. Studies have been conducted on inclusion of a drug into a micelle structure of an amphiphilic polymer for administration into the blood, and the resulting product has been under clinical trials (e.g., Y. Mizumura et al., Jap. J. Cancer Res., 93, 1237 (2002)).

In the cases of emulsified products, surfactant-induced electrostatic interactions are used, and this always causes stability problems, such as a droplet separation phenomenon. On the other hand, polymer micelles are structurally formed with covalent bonds, which is advantageous in terms of stability. Further, if miniaturization (nanoparticle formation) of polymer micelles can be achieved, sufficient transparency is obtained upon water dispersion. However, as compared with generally used synthetic surfactants, biodegradable polymers, and particularly, natural polymers such as proteins, are highly safe for use. Therefore, nanoparticles made of biodegradable polymers have been awaited.

Meanwhile, blood circulation promoters are widely added as skin-roughness-preventive, skin dietary supplement, or hair-growing/increasing components, to products such as cosmetics, including lotions, creams, and emulsions, quasi-drugs, and externally applied pharmaceutical products. They are categorized as synthetic substances, plant extracts, vitamins, sugars, or the like. However, such extracts are extracted from organic solvents such as ethanol and 1,3-butylene glycol. Thus, it has been known that it is not always possible to keep such extracts in a stable state when adding them to water dispersions. In addition, it has been known that products other than extracts are also very weakly water-soluble. Addition of such components can be achieved by controlling the contents of organic solvents from 20% to less than 100% or by emulsifying such components with surfactants, for example. However, it has been known that such organic solvents cause excessive skin degreasing, and that surfactants and the like induce skin irritation or allergy.

### SUMMARY OF THE INVENTION

It is an object of the present invention to solve the above problems of the conventional techniques. Specifically, it is an object of the present invention to provide a nanoparticle which comprises a blood circulation promoter and a biodegradable polymer, which is safe and excellent in terms of dispersion stability and has high transparency and good absorbability due to its small particle size.

As a result of intensive studies to achieve the above object, the present inventors have found that a water-dispersible nanoparticle can be prepared by mixing a blood circulation promoter with a biodegradable polymer. The present invention has been completed based on the above findings.

The present invention provides a water-dispersible nanoparticle which comprises a blood circulation promoter and a biodegradable polymer.

Preferably, the content of the blood circulation promoter is 0.1% to 100% by weight with respect to the weight of the biodegradable polymer.

Preferably, the average particle size is 10 to 1000 nm.

Preferably, the blood circulation promoter is an ionic substance or a fat-soluble substance.

Preferably, the blood circulation promoter is a cosmetic component, a functional-food component, a quasi-drug component, or a pharmaceutical product component.

Preferably, the blood circulation promoter is at least one blood circulation promoter selected from the group consisting of a tocophenol derivative, a nicotinic acid derivative, cephalanthin, finasteride, minoxidil, and a Swertia japonica extract.

Preferably, the biodegradable polymer is a protein.

Preferably, the protein is at least one protein selected from the group consisting of collagen, gelatin, acid-treated gelatin, albumin, ovalbumin, casein, sodium casein, transferrin, globulin, fibroin, fibrin, laminin, fibronectin, and vitronectin.

Preferably, the protein is subjected to crosslinking treatment during and/or after nanoparticle formation.

Preferably, a transglutaminase is used for the crosslinking treatment.

The present invention further provides a casein nanoparticle which is prepared by the following steps (a) to (c):
(a) mixing casein with a basic aqueous medium at a pH of from 8 to less than 11;
(b) adding at least one blood circulation promoter to the solution obtained in step (a); and
(c) injecting the solution obtained in step (b) into an acidic aqueous medium at a pH of 3.5 to 7.5.

The present invention further provides a casein nanoparticle which is prepared by the following steps (a) to (c):
(a) mixing casein with a basic aqueous medium at a pH of from 8 to less than 11;
(b) adding at least one blood circulation promoter to the solution obtained in step (a); and
(c) lowering the pH of the solution obtained in step (b) to a pH value that is pH 1 or more away from the isoelectric point, while stirring the solution.

The present invention further provides a drug delivery agent which comprises the nanoparticle of the present invention as mentioned above.

Preferably, the drug delivery agent of the present invention is used as a transdermally absorbable agent, a topical therapeutic agent, an oral therapeutic agent, an intradermal parenteral injection, a subcutaneous parenteral injection, an intramuscular parenteral injection, an intravenous parenteral injection, a cosmetic, a quasi-drug, a functional food, or a supplement.

The particle of the present invention which contains a blood circulation promoter is a nanoparticle, and thus it has good absorbability and high transparency. The nanoparticle of the present invention is a nanoparticle comprising a biodegradable polymer such as a protein, and thus the structure thereof is highly stable. In addition, the particle can be produced without using a chemical crosslinking agent or synthetic surfactant, and thus it is highly safe. Further, dispersion of nanoparticles containing a hydrophobic blood circulation promoter can be achieved. Thus, there is no need to add a large volume of ethanol, and therefore skin irritation caused by ethanol can be reduced.

### DESCRIPTION OF THE PREFERRED EMBODIMENTS

The embodiments of the present invention will be described in detail below.

The water-dispersible nanoparticle of the present invention is characterized in that it comprises a blood circulation promoter and a biodegradable polymer.

Specific examples of a blood circulation promoter that can be used in the present invention are described below, but they are not particularly limited thereto as long as blood circulation promoting effects can be exhibited. The term “blood circulation promotion” refers to effects of increasing the blood flow resulting from effects of, for example, blood vessel dilation induced by relaxation, enhancement and stimulation of capillary vessels, and temperature increase. Preferably, the blood circulation promoter used in the present invention is an ionic substance or fat-soluble substance. In addition, examples of the blood circulation promoter that can be used in the present invention include synthetic substances, plant extracts, vitamins, and sugars.

Examples of synthetic substances include minoxidil which is known to have effects of anti-hypertensive agents; finasteride which is known to have effects of anti-prostatic hypertrophy agents; and carpronium chloride which is known to have effects as a drug for alopecia areatas.

Examples of plant extracts include Swertia japonica extracts obtained from rhizomes and stolons of plants belonging to the family Gentianaceae, carrot extracts obtained from rhizomes and stolons of plants belonging to the family Araliaceae, Sophora angustifolia extracts obtained from rhizomes and stolons of plants belonging to the family Leguminosae, peppermint extracts obtained from peppermint leaves and the like, cepharanthine, which is an alkaloid of a plant belonging to the family Menispermaceae, cayenne pepper tinctures obtained from cayenne pepper, ginger tinctures obtained from ginger, and garlic extracts extracted from garlic.

Preferably, vitamins are vitamin B, vitamin E, and derivatives thereof. Examples of vitamin E and derivatives thereof include tocopherol, tocopherol acetate, and nicotinic acid tocopherol, which are preferably naturally occurring α-tocopherols. In addition, examples of vitamin B and derivatives thereof include nicotinic acid, nicotinic acid amide, and nicotinic acid benzyl, which are widely existing hydrophilic and hydrophobic substances.

Preferably, sugars are mucopolysaccharides. Specific examples thereof include heparin, which has a blood-coagulation-inhibiting action.

Preferably, the blood circulation promoter used in the present invention is a tocophenol derivative, a nicotinic acid derivative, cephalanthin, finasteride, minoxidil, or a Swertia japonica extract.

According to the present invention, a component used as the above blood circulation promoter can be selected from the group consisting of cosmetic components, quasi-drug components, functional-food components, and pharmaceutical product components. The blood circulation promoter used in the present invention may be used alone or in combinations of two or more.

According to the present invention, a blood circulation promoter may be added during, before or after the formation of nanoparticle of the biodegradable polymer.

The nanoparticle of the present invention preferably contains the blood circulation promoter in an amount of 0.1% to 100% by weight with respect to the weight of the biodegradable polymer, and more preferably contains the blood circulation promoter in an amount of 0.1% to 50% by weight with respect to the weight of the biodegradable polymer.

The average particle size of the nanoparticle of the present invention is generally 1 to 1000 nm, preferably 10 to 1000 nm, more preferably 10 to 500 nm, and particularly preferably 15 to 400 nm.

The biodegradable polymer used in the present invention may be a protein or a biodegradable synthetic polymer.

The type of the biodegradable polymer is not particularly limited. However, a protein having a lysine residue and a glutamine residue is preferable. In addition, such protein having a molecular weight of approximately 10,000 to 1,000,000 is preferably used. The origin of the protein is not particularly limited. However, a human-derived protein, is preferably used. Specific examples of a protein that can be used include at least one selected from the group consisting of collagen, gelatin, acid-treated gelatin, albumin, ovalbumin, casein, sodium casein, transferrin, globulin, fibroin, fibrin, laminin, fibronectin, and vitronectin. However, the compound used in the present invention is not limited to the aforementioned compounds. In addition, the origin of the protein is not particularly limited. Thus, bovine, swine, and fish, as well as recombinant protein of any thereof, can be used. Examples of recombinant gelatin that can be used include, but are not limited to, gelatins described in EP1014176 A2 and U.S. Pat. No. 6,992,172. Among them, casein, acid-treated gelatin, collagen, or albumin is preferable. Further, casein or acid-treated gelatin is most preferable. When casein is used in the present invention, the origin of the casein is not particularly limited. Casein may be milk-derived or bean-derived. Any of α-casein, β-casein, γ-casein, and κ-casein, as well as a mixture thereof, can be used. Caseins may be used alone or in combinations of two or more.

Proteins used in the present invention may be used alone or in combinations of two or more. Examples of the biodegradable synthetic polymer include polylactic acid, and poly(lactic-co-glycolic acid) (PLGA).

According to the present invention, a protein can be subjected to crosslinking treatment during and/or after nanoparticle formation. For the crosslinking treatment, an enzyme can be used. Any enzyme may be used without particular limitation as long as it has been known to have an action of causing protein crosslinking. Among such enzymes, transglutaminase is preferable.

Transglutaminase may be derived from a mammal or a microorganism. A recombinant transglutaminase can be used. Specific examples thereof include the Activa series by Ajinomoto Co., Inc., commercially available mammalian-derived transglutaminase serving as a reagent, such as guinea pig liver-derived transglutaminase, goat-derived transglutaminase, rabbit-derived transglutaminase, or human-derived recombinant transglutaminase produced by, for example, Oriental Yeast Co., Ltd., Upstate USA Inc., and Biodesign International.

The amount of an enzyme used for the crosslinking treatment in the present invention can be adequately determined depending upon protein type. In general, an enzyme can be added in a weight that is 0.1% to 100% and preferably approximately 1% to 50% of the protein weight.

The duration for an enzymatic crosslinking reaction can be adequately determined depending upon protein type and nanoparticle size. However, in general, the reaction can be carried out for 1 to 72 hours, and preferably 2 to 24 hours.

The temperature for an enzymatic crosslinking reaction can be adequately determined depending upon protein type and nanoparticle size. In general, the reaction can be carried out at 0° C. to 80° C. and preferably at 25° C. to 60° C.

Enzymes used in the present invention may be used alone or in combinations of two or more.

Nanoparticles of the present invention can be prepared in accordance with Patent Document: JP Patent Publication (Kokai) No. 6-79168 A (1994); or C. Coester, Journal Microcapsulation, 2000, vol. 17, pp. 187-193, provided that an enzyme is preferably used instead of glutaraldehyde for a crosslinking method.

In addition, according to the present invention, the enzymatic crosslinking treatment is preferably carried out in an organic solvent. The organic solvent used herein is preferably an aqueous organic solvent such as ethanol, isopropanol, acetone, or THF.

It is also possible to add at least one component selected from the group consisting of lipids (e.g., phospholipid), anionic polysaccharides, cationic polysaccharides, anionic proteins, cationic proteins, and cyclodextrin to the water-dispersible nanoparticle of the present invention. The amounts of lipid (e.g. phospholipid), anionic polysaccharide, cationic polysaccharide, anionic protein, cationic protein, and cyclodextrin to be added are not particularly limited. However, they can be added usually in a weight that is 0.1% to 100% of the protein weight. In the case of the drug delivery agent of the present invention, it is possible to adjust the release rate by changing the ratio of the above components to the protein.

Specific examples of phospholipids that can be used in the present invention include, but are not limited to, the following compounds: phosphatidylcholine (lecithin), phosphatidylethanolamine, phosphatidylserine, phosphatidylinositol, phosphatidylglycerol, diphosphatidylglycerol, and sphingomyelin.

Anionic polysaccharides that can be used in the present invention are polysaccharides having an acidic polar group such as a carboxyl group, a sulfate group, or a phosphate group. Specific examples thereof include, but are not limited to, the following compounds: chondroitin sulfate, dextran sulfate, carboxymethyl cellulose, carboxymethyl dextran, alginic acid, pectin, carrageenan, fucoidan, agaropectin, porphyran, karaya gum, gellan gum, xanthan gum, and hyaluronic acids.

Cationic polysaccharides that can be used in the present invention are polysaccharides having a basic polar group such as an amino group. Examples thereof include, but are not limited to, the following compounds: polysaccharides such as chitin or chitosan, which comprise, as a monosaccharide unit, glucosamine or galactosamine.

Anionic proteins that can be used in the present invention are proteins and lipoproteins having a more basic isoelectric point than the physiological pH. Specific examples thereof include, but are not limited to, the following compounds: polyglutamic acid, polyaspartic acid, lysozyme, cytochrome C, ribonuclease, trypsinogen, chymotrypsinogen, and α-chymotrypsin.

Cationic proteins that can be used in the present invention are proteins and lipoproteins having a more acidic isoelectric point than the physiological pH. Specific examples thereof include, but are not limited to, the following compounds: polylysine, polyarginine, histone, protamine, and ovalbumin.

According to the present invention, it is possible to use casein nanoparticles prepared by the following steps (a) to (c):
(a) mixing casein with a basic aqueous medium at a pH of from 8 to less than 11;
(b) adding at least one blood circulation promoter to the solution obtained in step (a); and
(c) injecting the solution obtained in step (b) into an acidic aqueous medium at a pH of 3.5 to 7.5.

Further, according to the present invention, it is possible to use casein nanoparticles prepared by the following steps (a) to (c):
(a) mixing casein with a basic aqueous medium at a pH of from 8 to less than 11;
(b) adding at least one blood circulation promoter to the solution obtained in step (a); and
(c) lowering the pH of the solution obtained in step (b) to a pH value that is pH 1 or more away from the isoelectric point, while stirring the solution.

According to the present invention, it is possible to prepare casein nanoparticles of desired sizes. Also, with the use of interaction between a hydrophobic blood circulation promoter and a casein hydrophobic domain, it is possible for casein nanoparticles to contain the blood circulation promoter. In addition, it was found that such particles remain stable in an aqueous solution.

Further, it was found that a particle mixture of casein and ionic polysaccharide or another ionic protein contains an ionic blood circulation promoter.

The method for preparing casein nanoparticles of the present invention involves a method wherein casein is mixed with a basic aqueous medium solution and the solution is injected into another acidic aqueous medium, and a method wherein casein is mixed with a basic aqueous medium solution and the pH of the solution is lowered during stirring, for example.

The method wherein casein is mixed with a basic aqueous medium solution and the solution is injected into another acidic aqueous medium is preferably carried out using a syringe for convenience. However, there is no particular limitation as long as the injection rate, solubility, temperature, and stirring conditions are satisfied. Injection can be carried out usually at an injection rate of 1 mL/min to 100 mL/min. The temperature of the basic aqueous medium can be adequately determined. In general, the temperature is 0° C. to 80° C. and preferably 25° C. to 70° C. The temperature of an acidic aqueous medium can be adequately determined. In general, the temperature can be 0° C. to 80° C. and preferably 25° C. to 60° C. The stirring rate can be adequately determined. However, in general, the stirring rate can be 100 rpm to 3000 rpm and preferably 200 rpm to 2000 rpm.

In the method wherein casein is mixed with a basic aqueous medium solution and the pH of the medium is lowered during stirring, it is preferable to add acid dropwise for convenience. However, there is no particular limitation as long as solubility, temperature, and stirring conditions are satisfied. The temperature of a basic aqueous medium can be adequately determined. However, in general, the temperature can be 0° C. to 80° C. and preferably 25° C. to 70° C. The stirring rate can be adequately determined. However, in general, the stirring rate can be 100 rpm to 3000 rpm and preferably 200 rpm to 2000 rpm.

The aqueous medium that can be used for the present invention is an aqueous solution or a buffer comprising an organic acid or base or an inorganic acid or base.

Specific examples thereof include, but are not limited to, aqueous solutions comprising: organic acids such as citric acid, ascorbic acid, gluconic acid, carboxylic acid, tartaric acid, succinic acid, acetic acid, phthalic acid, trifluoroacetic acid, morpholinoethanesulfonic acid, and 2-[4-(2-hydroxyethyl)-1-piperazinyl]ethanesulfonic acid; organic bases such as tris (hydroxymethyl), aminomethane, and ammonia; inorganic acids such as hydrochloric acid, perchloric acid, and carbonic acid; and inorganic bases such as sodium phosphate, potassium phosphate, calcium hydroxide, sodium hydroxide, potassium hydroxide, and magnesium hydroxide.

The concentration of an aqueous medium used in the present invention is preferably approximately 10 mM to 1 M, and more preferably approximately 20 mM to 200 mM.

The pH of a basic aqueous medium used in the present invention is preferably 8 or more and less than 11, and more preferably 10 to 11. When the pH is excessively high, there is concern regarding hydrolysis or risks in handling. Thus, the pH is preferably in the above range.

According to the present invention, the temperature at which casein is mixed with a basic aqueous medium at pH of 8 or more and less than 11 is preferably 0° C. to 90° C., more preferably 10° C. to 80° C., and further preferably 20° C. to 70° C.

The pH of an acidic aqueous medium used in the present invention is preferably 3.5 to 7.5 and more preferably 5 to 6. If the pH is beyond the aforementioned range, there is a tendency where the particle size becomes large.

The nanoparticle of the present invention comprises a blood circulation promoter. When the blood circulation promoter is an active component, the nanoparticle of the present invention which comprises such an active component can be administered to the affected part for use. Specifically, the nanoparticle of the present invention is useful as a drug delivery agent.

Preferably, the nanoparticle of the present invention is administered via transdermal or transmucosal absorption, or injection into blood vessel, body cavity or lympho tissue. More preferably, the nanoparticle of the present invention is administered via transdermal or transmucosal absorption.

In the present invention, the usage of the drug delivery agent is not particularly limited. For example, the drug delivery agent is used as transdermally absorbable agent, a topical therapeutic agent, an oral therapeutic agent, an intradermal parenteral injection, a subcutaneous parenteral injection, an intramuscular parenteral injection, an intravenous parenteral injection, a cosmetic, a quasi-drug, a functional food, or a supplement.

In the present invention, the drug delivery agent may comprise an additive. The type of such additive is not particularly limited. Examples of such additive include a moisturizer, a softener, an antiinflammatory agent, a percutaneous absorption promoter, soothing agents, preservatives, antioxidants, coloring agents, thickeners, aroma chemicals, and pH adjusters.

Specific examples of the moisturizer that can be used in the present invention include, but are not limited to, agar, diglycerin, distearyldimonium hectorite, butylene glycol, polyethylene glycol, propylene glycol, hexylene glycol, coix seed extract, vaserine, urea, hyaluronic acid, ceramide, Lipidure, isoflavone, amino acid, collagen, mucopolysaccharide, fucoidan, lactoferrin, sorbitol, chitin, chitosan, malic acid, glucuronic acid, Placenta extract, Seaweed extract, Moutan cortex extract, Hydrangeae dulcis folium extract, hypericum extract, coleus extract, Euonymus japonica, safflower extract, Rosa rugosa flower extract, Polyporus Sclerotium extract, hawthorn extract, rosemary extract, duku extract, chamomile extract, lamium album extract, Litchi Chinensis extract, Achillea Millefolium extract, aloe extract, marronnier extract, Thujopsis dolabrata extract, Fucus extract, Osmoin extract, oat extract, Tuberosa polysaccharide, Cordyceps Sinensis extract, barley extract, orange extract, Rehmannia root extract, zanthoxylum fruit extract, and coix seed extract.

Specific examples of the softener that can be used in the present invention include, but are not limited to, glycerin, mineral oil, and emollient ingredients (e.g. isopropyl isostearate, polyglyceryl isostearate, isotridecyl isononanoate, octyl isononanoate, oleic acid, glyceryl oleate, cacao butter, cholesterol, mixed fatty acid triglyceride, dioctyl succinate, sucrose acetate stearate, cyclopentanesiloxane, sucrose distearate, octyl palmitate, octyl hydroxystearate, arachidyl behenate, sucrose polybehenate, polymethylsilsesquioxane, myristyl alcohol, cetyl myristate, myristyl myristate, and hexyl laurate).

Examples of an antiinflammatory agent used in the present invention may include a compound which is selected from azulene, guaiazulene, diphenhydramine hydrochloride, hydrocortisone acetate, prednisolone, glycyrrhizinic acid, glycyrrhetinic acid, mefenamic acid, phenylbutazone, indometacin, ibuprofen and ketoprofen, and its derivative and its salt; and a plant extract which is selected from Scutellariae Radix extract, Artemisia capillaris Thunb. Extract, Platycodon grandiflorum extract, Armeniacae Semen extract, Common gardenia extract, Sasa veitchii extract, Gentiana lutea extract, Comfrey extract, white birch extract, Malva extract, Persicae Semen extract, peach blade extract, and loquat blade extract; proteins; polysaccharides; and animal extracts, but are not limited thereto.

Specific examples of the percutaneous absorption promoter that can be used in the present invention include, but are not limited to, ethanol, isopropyl myristate, citric acid, squalane, oleic acid, menthol, N-methyl-2-pyrrolidone, diethyl adipate, diisopropyl adipate, diethyl sebacate, diisopropyl sebacate, isopropyl palmitate, isopropyl oleate, octyldodecyl oleate, isostearyl alcohol, 2-octyldodecanol, urea, vegetable oil, and animal oil.

Specific examples of soothing agents that can be used in the present invention include, but are not limited to, the following compounds: benzyl alcohol, procaine hydrochloride, xylocaine hydrochloride, and chlorobutanol.

Specific examples of preservatives that can be used in the present invention include, but are not limited to, the following compounds: benzoic acid, sodium benzoate, paraben, ethylparaben, methylparaben, propylparaben, butylparaben, potassium sorbate, sodium sorbate, sorbic acid, sodium dehydroacetate, hydrogen peroxide, formic acid, ethyl formate, sodium hypochlorite, propionic acid, sodium propionate, calcium propionate, pectin degradation products, polylysine, phenol, isopropylmethyl phenol, orthophenylphenol, phenoxyethanol, resorcin, thymol, thiram, and tea tree oil.

Specific examples of antioxidants that can be used in the present invention include, but are not limited to, the following compounds: vitamin A, retinoic acid, retinol, retinol acetate, retinol palmitate, retinyl acetate, retinyl palmitate, tocopheryl retinoate, vitamin C and derivatives thereof, kinetin, β-carotene, astaxanthin, lutein, lycopene, tretinoin, vitamin E, α-lipoic acid, coenzyme Q10, polyphenol, SOD, and phytic acid.

Specific examples of coloring agents that can be used in the present invention include, but are not limited to, the following compounds: krill pigment, orange dye, cacao dye, kaoline, carmines, ultramarine blue, cochineal dye, chrome oxide, iron oxide, titanium dioxide, tar dye, and chlorophyll.

Specific examples of thickeners that can be used in the present invention include, but are not limited to, the following compounds: quince seed, carrageenan, gum arabic, karaya gum, xanthan gum, gellan gum, tamarind gum, locust bean gum, gum traganth, pectin, starch, cyclodextrin, methylcellulose, ethylcellulose, carboxymethylcellulose, sodium alginate, polyvinyl alcohol, polyvinyl pyrrolidone, carboxyvinyl polymer, and sodium polyacrylate.

Specific examples of aroma chemicals that can be used in the present invention include, but are not limited to, the following compounds: musk, acacia oil, anise oil, ylang ylang oil, cinnamon oil, jasmine oil, sweet orange oil, spearmint oil, geranium oil, thyme oil, neroli oil, mentha oil, hinoki (Japanese cypress) oil, fennel oil, peppermint oil, bergamot oil, lime oil, lavender oil, lemon oil, lemongrass oil, rose oil, rosewood oil, anisaldehyde, geraniol, citral, civetone, muscone, limonene, and vanillin.

Specific examples of pH adjusters that can be used in the present invention include, but are not limited to, the following compounds: sodium citrate, sodium acetate, sodium hydroxide, potassium hydroxide, phosphoric acid, and succinic acid.

The dose of the nanoparticle of the present invention can be adequately determined depending upon type and amount of active ingredient and upon user weight and condition, for example. The dose for single administration is generally approximately 10 μg to 100 mg/kg, and preferably 20 μg to 50 mg/kg. In case of via transdermal or transmucosal administration, the nanoparticle can be administered in an amount of approximately 1 μg to 50 mg/cm, and preferably 2.5 μg to 10 mg/cm.

The present invention is hereafter described in greater detail with reference to the following examples, although the technical scope of the present invention is not limited thereto.

### EXAMPLES

### Example 1

Milk-derived casein Na (10 mg; Wako Pure Chemical Industries, Ltd.) was mixed with 50 mM phosphate buffer (pH 9)(1 mL). Tocopherol acetate (0.75 mg; Wako Pure Chemical Industries, Ltd.) was dissolved in ethanol (0.1 mL). The tocopherol acetate solution was added dropwise to the casein solution during stirring. The resulting liquid mixture (1 ml) was injected into 200 mM phosphate buffer water (pH 5) (10 mL) with the use of a microsyringe at an external temperature of 40° C. during stirring at 800 rpm. Thus, a water dispersion of casein nanoparticles containing tocopherol acetate was obtained. The particle size of the obtained casein particles was measured with a “Zetasizer Nano” (Sysmex), and the volume average particle size was determined. It was found to be 18.0 nm.

### Example 2

Nanoparticles were prepared as in Example 1, except that tocopherol acetate (3.75 mg; Wako Pure Chemical Industries, Ltd.) was dissolved in ethanol (0.1 mL). The particle size of the obtained particles was measured with a “Zetasizer Nano” (Sysmex), and the volume average particle size was determined. It was found to be 19.2 nm.

### Example 3

Nanoparticles were prepared as in Example 1, except that tocopherol nicotinate (0.5 mg; Wako Pure Chemical Industries, Ltd.) was dissolved in ethanol (0.1 mL). The particle size of the obtained particles was measured with a “Zetasizer Nano” (Sysmex), and the volume average particle size was determined. It was found to be 19.2 nm.

### Example 4

Nanoparticles were prepared as in Example 1, except that tocopherol nicotinate (2.5 mg; Wako Pure Chemical Industries, Ltd.) was dissolved in ethanol (0.1 mL). The particle size of the obtained particles was measured with a “Zetasizer Nano” (Sysmex), and the volume average particle size was determined. It was found to be 20.5 nm.

### Example 5

Nanoparticles were prepared as in Example 1, except that tocopherol (0.75 mg; Wako Pure Chemical Industries, Ltd.) was dissolved in ethanol (0.1 mL). The particle size of the obtained particles was measured with a “Zetasizer Nano” (Sysmex), and the volume average particle size was determined. It was found to be 18.8 nm.

### Example 6

Nanoparticles were prepared as in Example 1, except that tocopherol (3.75 mg; Wako Pure Chemical Industries, Ltd.) was dissolved in ethanol (0.1 mL). The particle size of the obtained particles was measured with a “Zetasizer Nano” (Sysmex), and the volume average particle size was determined. It was found to be 20.3 nm.

### Example 7

Nanoparticles were prepared as in Example 1, except that nicotinic-acid amide (0.85 mg; Wako Pure Chemical Industries, Ltd.) was dissolved in ethanol (0.2 mL). The particle size of the obtained particles was measured with a “Zetasizer Nano” (Sysmex), and the volume average particle size was determined. It was found to be 20.0 nm.

### Example 8

Nanoparticles were prepared as in Example 1, except that benzyl nicotinate (1.1 mg; Wako Pure Chemical Industries, Ltd.) was dissolved in ethanol (0.1 mL). The particle size of the obtained particles was measured with a “Zetasizer Nano” (Sysmex), and the volume average particle size was determined. It was found to be 17.5 nm.

### Example 9

Nanoparticles were prepared as in Example 1, except that cephalanthin (0.85 mg; Wako Pure Chemical Industries, Ltd.) was dissolved in ethanol (0.3 mL). The average particle size of the obtained particles was measured with a light scattering photometer (Microtrack, Nikkiso, Co., Ltd.), and was found to be 22 nm.

### Example 10

Nanoparticles were prepared as in Example 1, except that finasteride (0.85 mg; LKT Laboratories Inc.) was dissolved in ethanol (0.3 mL). The average particle size of the obtained particles was measured with a light scattering photometer (Microtrack, Nikkiso, Co., Ltd.), and was found to be 29 nm.

### Example 11

Nanoparticles were prepared as in Example 1, except that minoxidil (1 mg; LKT Laboratories Inc.) was dissolved in ethanol (0.02 mL). The average particle size of the obtained particles was measured with a light scattering photometer (Microtrack, Nikkiso, Co., Ltd.), and was found to be 23.4 nm.

### Example 12

Nanoparticles were prepared as in Example 1, except that Swertia japonica extract (1.05 ml; Maruzen Pharmaceuticals Co., Ltd.; Swertia japonica extract liquid) was added dropwise. The particle size of the obtained particles was measured with a “Zetasizer Nano” (Sysmex), and the volume average particle size was determined. It was found to be 26.3 nm.

### Example 13

Nanoparticles were prepared as in Example 1, except that benzyl nicotinate (1.1 mg; Wako Pure Chemical Industries, Ltd.) was dissolved in ethanol (0.1 mL) and chili pepper tincture (0.01 ml; Maruzen Pharmaceuticals Co., Ltd.) was added dropwise. The particle size of the obtained particles was measured with a “Zetasizer Nano” (Sysmex), and the volume average particle size was determined. It was found to be 27.3 nm.

### Example 14

Nanoparticles were prepared as in Example 1, except that collagen (Nitta Gelatin Inc.), gelatin, acid-treated gelatin, or albumin was used in place of casein. As a result, similar nanoparticles were obtained.

### Test Example 1

Water dispersions of casein nanoparticles for Examples 8 and 13 and water dispersions obtained by removing casein from those used in Examples 8 and 13 for Comparative Examples (referred to as Comparative Example 8A and Comparative Example 13A, respectively) were prepared and allowed to stand at 4° C. for 16 hour. Precipitation was exclusively observed in the water dispersions for Comparative Examples 8A and 13A, while on the other hand, no precipitation was observed in the water dispersions for Examples 8 and 13. The results indicate that the nanoparticles of the present invention are excellent in terms of stability.

### Test Example 2

Dorsal hair of C3H mice at the trichogenous or dormant phase were cut with a hair clipper. On the next day, the mice were shaved with a shaver. The water dispersions of protein nanoparticles containing a blood circulation promoter which were prepared in Examples 8 and 9 were separately applied to all shaved areas once daily. The degree of ability to cause phase transition to the growth phase in mouse dorsal hair follicles was examined. As a result, hair growth effects were promoted and activity of causing hair cycle transition from the dormant phase to the growth phase was observed, as compared with the cases of ethanol solutions containing a blood circulation promoter alone (at the same concentrations) used in Examples 8 and 9. Therefore, it has been revealed that the water dispersions of the present invention cause no excessive skin degreasing or skin irritation caused by an ethanol solution, and exhibit hair growth effects derived from blood circulation promoting effects to a greater extent than the case of using ethanol solution.

1. A water-dispersible nanoparticle which comprises a blood circulation promoter and a biodegradable polymer.

2. The nanoparticle according to claim 1, wherein the content of the blood circulation promoter is 0.1% to 100% by weight with respect to the weight of the biodegradable polymer.

3. The nanoparticle according to claim 1, wherein the average particle size is 10 to 1000 nm.

4. The nanoparticle according to claim 1, wherein the blood circulation promoter is an ionic substance or a fat-soluble substance.

5. The nanoparticle according to claim 4, wherein the blood circulation promoter is a cosmetic component, a functional-food component, a quasi-drug component, or a pharmaceutical product component.

6. The nanoparticle according to claim 1, wherein the blood circulation promoter is at least one blood circulation promoter selected from the group consisting of a tocophenol derivative, a nicotinic acid derivative, cephalanthin, finasteride, minoxidil, and a Swertia japonica extract.

7. The nanoparticle according to claim 1, wherein the biodegradable polymer is a protein.

8. The nanoparticle according to claim 7, wherein the protein is at least one protein selected from the group consisting of collagen, gelatin, acid-treated gelatin, albumin, ovalbumin, casein, sodium casein, transferrin, globulin, fibroin, fibrin, laminin, fibronectin, and vitronectin.

9. The nanoparticle according to claim 7, wherein the protein is subjected to crosslinking treatment during and/or after nanoparticle formation.

10. The nanoparticle according to claim 9, wherein a transglutaminase is used for the crosslinking treatment.

11. A casein nanoparticle which is prepared by the following steps (a) to (c):
(a) mixing casein with a basic aqueous medium at a pH of from 8 to less than 11;
(b) adding at least one blood circulation promoter to the solution obtained in step (a); and
(c) injecting the solution obtained in step (b) into an acidic aqueous medium at a pH of 3.5 to 7.5.

12. A casein nanoparticle which is prepared by the following steps (a) to (c):
(a) mixing casein with a basic aqueous medium at a pH of from 8 to less than 11;
(b) adding at least one blood circulation promoter to the solution obtained in step (a); and
(c) lowering the pH of the solution obtained in step (b) to a pH value that is pH 1 or more away from the isoelectric point, while stirring the solution.

13. A drug delivery agent which comprises the nanoparticle of claim 1.

14. The drug delivery agent according to claim 13, which is used as a transdermally absorbable agent, a topical therapeutic agent, an oral therapeutic agent, an intradermal parenteral injection, a subcutaneous parenteral injection, an intramuscular parenteral injection, an intravenous parenteral injection, a cosmetic, a quasi-drug, a functional food, or a supplement.
